(19) United States
(12) Patent Application Publication
(10) Pub. No.: US 2005/0170430 A1
(43) Pub. Date: Aug. 4, 2005

(54) Diagnosis and treatment of glaucoma and methods for discovering new glaucoma therapeutic agents based on the wnt/ca2+ signaling pathway

(76) Inventors: Abbot F Clark, Arlington, TX (US); Wan-Heng Wang, Grapevine, TX (US); Loretta Graves McNatt, Hurst, TX (US)

Correspondence Address: Alcon Research, 6201 South Freeway, Fort Worth, TX, 76134-2099, US

(21) Appl. No.: 10/512,513

(22) PCT Filed: Apr. 30, 2003
(86) PCT No.: PCT/US03/13384

Related U.S. Application Data
(60) Provisional application No. 60/377,485, filed on May 3, 2002.

(51) Int. Cl. C12Q 1/68; G01N 33/53

(52) U.S. Cl. 435/7.1; 435/6

(57) ABSTRACT

The present invention provides methods for diagnosing and treating glaucoma and identifying agents potentially useful for treating glaucoma. The invention further provides compositions useful for treating glaucoma.

### BACKGROUND OF THE INVENTION

1. Field of the Invention

The present invention relates to the field of diagnosis and treatment of glaucoma. More specifically, the invention provides methods and compositions for diagnosing and is treating glaucoma and for identifying agents potentially useful for the treatment of glaucoma.

2. Description of the Related Art

There are a number of ocular conditions that are caused by, or aggravated by, damage to the optic nerve head, degeneration of ocular tissues, and/or elevated intraocular pressure. For example, “glaucomas” are a group of debilitating eye diseases that are a leading cause of irreversible blindness in the United States and other developed nations. Primary Open Angle Glaucoma (“POAG”) is the most common form of glaucoma. The disease is characterized by the degeneration of the trabecular meshwork, leading to obstruction of the normal ability of aqueous humor to leave the eye without closure of the space (e.g., the “angle”) between the iris and cornea (Vaughan, D. et al., (1992)). A characteristic of such obstruction in this disease is an increased intraocular pressure (“IOP”), resulting in progressive visual loss and blindness if not treated appropriately and in a timely fashion. The disease is estimated to affect between 0.4% and 3.3% of all adults over 40 years old (Leske, M. C. et al. (1986); Bengtsson, B. (1989); Strong, N. P. (1992)). Moreover, the prevalence of the disease rises with age to over 6% of those 75 years or older (Strong, N. P., (1992)).

Glaucoma affects three separate tissues in the eye. The elevated IOP associated with POAG is due to morphological and biochemical changes in the trabecular meshwork (TM), a tissue located at the angle between the cornea and iris. Most of the nutritive aqueous humor exits the anterior segment of the eye through the TM. The progressive loss of TM cells and the build-up of extracellular debris in the TM of glaucomatous eyes leads to increased resistance to aqueous outflow, thereby raising IOP. Elevated IOP, as well as other factors such as ischemia, cause degenerative changes in the optic nerve head (ONH) leading to progressive “cupping” of the ONH and loss of retinal ganglion cells and axons. The detailed molecular mechanisms responsible for glaucomatous damage to the TM, ONH, and the retinal ganglion cells are unknown.

Twenty years ago, the interplay of ocular hypertension, ischemia and mechanical distortion of the optic nerve head were heavily debated as the major factors causing progression of visual field loss in glaucoma. Since then, other factors including excitotoxicity, nitric oxide, absence of vital neurotrophic factors, abnormal glial/neuronal interplay and genetics have been implicated in the degenerative disease process. The consideration of molecular genetics deserves some discussion insofar as it may ultimately define the mechanism of cell death, and provide for discrimination of the various forms of glaucoma. Within the past 8 years, over 15 different glaucoma genes have been mapped and 7 glaucoma genes identified. This includes six mapped genes (GLC1A-GLC1F) and two identified genes (MYOC and OPTN) for primary open angle glaucoma, two mapped genes (GLC3A-GLC3B) and one identified gene for congentical glaucoma (CYP1B1), two mapped genes for pigmentary dispersion/pigmentary glaucoma, and a number of genes for developmental or syndromic forms of glaucoma (FOXC1, PITX2, LAM1B, PAX6).

Thus, each form of glaucoma may have a unique pathology and accordingly a different therapeutic approach to the management of the disease may be required. For example, a drug that effects the expression of enzymes that degrade the extracellular matrix of the optic nerve head would not likely prevent RGC death caused by excitotoxicity or neurotrophic factor deficit. In glaucoma, RGC death occurs by a process called apoptosis (programmed cell death). It has been speculated that different types of insults that can cause death may do so by converging on a few common pathways. Targeting downstream at a common pathway is a strategy that may broaden the utility of a drug and increase the probability that it may have utility in the management of different forms of the disease. However, drugs that effect multiple metabolic pathways are more likely to produce undesirable side-effects. With the advent of gene-based diagnostic kits to identify specific forms of glaucoma, selective neuroprotective agents can be tested with the aim of reducing the degree of variation about the measured response.

Glaucoma is currently diagnosed based on specific signs of the disease (characteristic optic nerve head changes and visual field loss). However, over half of the population with glaucoma are unaware they have this blinding disease and by the time they are diagnosed, they already have irreversibly lost approximately 30-50% of their retinal ganglion cells. Thus, improved methods for early diagnosis of glaucoma are needed.

Current glaucoma therapy is directed to lowering IOP, a major risk factor for the development and progression of glaucoma. However, none of the current IOP lowering therapies actually intervenes in the glaucomatous disease process responsible for elevated IOP and progressive damage to the anterior segment continues. This is one possible reason why most patients become “resistant” to conventional glaucoma therapies. Thus, what is needed is a therapeutic method for altering (by inhibiting or even reversing) the disease process.

### SUMMARY OF THE INVENTION

The present invention overcomes these and other drawbacks of the prior art by providing a method for diagnosing glaucoma in a patient by detecting the level or bioactivity of Wnt/Ca²⁺ pathway component, frizzled related protein gene product of the Wnt/Ca²⁺ pathway, or an FRP of the Wnt/Ca²⁺ pathway in a sample obtained from the patient and comparing the level or bioactivity of Wnt/Ca²⁺ pathway component, frizzled related protein gene product of the Wnt/Ca²⁺ pathway, or FRP of the Wnt/Ca²⁺ pathway with the level in a normal sample. An aberrant level or bioactivity of Wnt/Ca²⁺ pathway component, frizzled related protein gene product of the Wnt/Ca²⁺ pathway, or FRP of the Wnt/Ca²⁺ pathway as compared to the level in a normal sample is indicative of a glaucomatous state. Preferably, the sample from the patient will include cells of the trabecular meshwork tissue or patient tears.

In another aspect, the invention provides a method for diagnosing glaucoma in a patient by isolating a Wnt/Ca²⁺ pathway component, a frizzled related protein gene product of the Wnt/Ca²⁺ pathway, or an FRP of the Wnt/Ca²⁺ pathway from a sample obtained from the patient and comparing the sequence of Wnt/Ca²⁺ pathway component, frizzled related protein gene product of the Wnt/Ca²⁺ pathway, or FRP of the Wnt/Ca²⁺ pathway with the sequence of a wildtype Wnt/Ca²⁺ pathway component, frizzled related protein gene product of the Wnt/Ca²⁺ pathway, or FRP of the Wnt/Ca²⁺ pathway. The presence of a genetic lesion in the sequence of Wnt/Ca²⁺ pathway component, frizzled related protein gene product of the Wnt/Ca²⁺ pathway, or FRP of the Wnt/Ca²⁺ pathway obtained from the sample as compared to the wildtype sequence indicates a glaucomatous state.

In yet another embodiment, the present invention provides a method of identifying an agent potentially useful for treating glaucoma by contacting a cell expressing Wnt/Ca²⁺ pathway component with a candidate substance, detecting a level or bioactivity of the Wnt/Ca²⁺ pathway component in the presence of the candidate substance, and comparing the level or bioactivity of the Wnt/Ca²⁺ pathway component in the presence of the candidate substance with that in the absence of the candidate substance. Typcially, an increase in the level or bioactivity of the Wnt/Ca²⁺ pathway component in the presence of the candidate substance as compared to the level or bioactivity detected in the absence of the candidate substance identifies the candidate substance as an agent potentially useful for treating glaucoma.

Alternatively, the present invention provides a method of identifying an agent potentially useful for treating glaucoma by admixing a composition containing a Wnt/Ca²⁺ pathway component polypeptide with a candidate substance, adding a composition containing a Wnt/Ca²⁺ pathway component binding partner to the first solution under conditions conducive to allow binding of the Wnt/Ca²⁺ pathway component polypeptide to the Wnt/Ca²⁺ pathway component binding partner, detecting the interaction of the Wnt/Ca²⁺ pathway component polypeptide with the binding partner in the presence of the candidate substance and in the absence of the candidate substance, and comparing the interaction of the Wnt/Ca²⁺ pathway component polypeptide and the binding partner in the presence of the candidate substance with that in the absence of the candidate substance. Depending upon the identities of the Wnt/Ca²⁺ pathway component and binding partner, an increase or decrease in the interaction of the Wnt/Ca²⁺ pathway component polypeptide with the binding partner in the presence of the candidate substance as compared to that in the absence of the candidate substance identifies the candidate as an agent potentially useful for treating glaucoma.

In preferred embodiments, the Wnt/Ca²⁺ pathway component may be LRP, Fzd, hteromeric G protein, PLC, PKC, CamKII or FRP.

Another embodiment of the present invention provides a method for treating glaucoma in a patient by administering to the patient a composition comprising a therapeutically effective amount of a compound that modulates the level or bioactivity of a Wnt/Ca²⁺ pathway component, a frizzled related protein gene product of the Wnt/Ca²⁺ pathway, or an FRP of the Wnt/Ca²⁺ pathway. In preferred embodiments, the compound may be a protein, a peptide, a peptidomimetic, a small molecule or a nucleic acid. Most preferably, the compound is a nucleic acid, such as a gene, antisense, ribozyme or triplex nucleic acid.

The present invention further provides a composition for treating glaucoma comprising a therapeutically effective amount of a compound that modulates the level or bioactivity of a Wnt/Ca²⁺ pathway component, a frizzled related protein gene product of the Wnt/Ca²⁺ pathway, or an FRP of the Wnt/Ca²⁺ pathway.

### DETAILED DESCRIPTION PREFERRED EMBODIMENTS

Glaucoma is a heterogeneous group of optic neuropathies that share certain clinical features. The loss of vision in glaucoma is due to the selective death of retinal ganglion cells in the neural retina that is clinically diagnosed by characteristic changes in the visual field, nerve fiber layer defects, and a progressive cupping of the ONH. One of the main risk factors for the development of glaucoma is the presence of ocular hypertension (elevated intraocular pressure, IOP). IOP also appears to be involved in the pathogenesis of normal tension glaucoma where patients have what is often considered to be normal IOP. The elevated IOP associated with glaucoma is due to elevated aqueous humor outflow resistance in the trabecular meshwork (TM), a small specialized tissue located in the iris-corneal angle of the ocular anterior chamber. Glaucomatous changes to the TM include a loss in TM cells and the deposition and accumulation of extracellular debris including plaque-like material. In addition, there also are changes that occur in the glaucomatous optic nerve head. In glaucomatous eyes, there are morphological and mobility changes in ONH glial cells. In response to elevated IOP and/or transient ischemic insults, there is a change in the composition of the ONH extracellular matrix and alterations in the glial cell and retinal ganglion cell axon morphologies.

The Wnt gene family encodes secreted ligand proteins that serve key roles in differentiation and development. This family comprises at least 15 vertebrate and invertebrate genes, including the Drosophila segment polarity gene, wingless, and one of its vertebrate homologues, integrated, from which the Wnt name derives. The Wnt proteins appear to facilitate a number of developmental and homeostatic processes. For example, vertebrate Wnt1 appears to be active in inducing myotome formation within the somites and in establishing the boundaries of the midbrain (McMahon and Bradley 1990; Ku and Melton 1993; Stern et al. 1995). During mammalian gastrulation, Wnt3a, Wnt5a, and Wnt5b are expressed in distinct yet overlapping regions within the primitive streak. Wnt3a is the only Wnt protein seen in the regions of the streak that will generate the dorsal (somite) mesoderm, and mice homozygous for a null allele of the Wnt3a gene have no somites caudal to the forelimbs. The Wnt genes also are important in establishing the polarity of vertebrate limbs, just as the invertebrate homolog wingless has been shown to establish polarity during insect limb development. In both cases there are interactions with Hedgehog family members as well.

There are three known Wnt signaling pathways (Miller 2001). The most extensively studied Wnt signaling pathway is the canonical Wnt/β-catenin pathway. The present inventors have discovered that the β-catenin pathway is present in the TM. This finding is the subject of U.S. application Ser. No. 09/796,008. That application describes only the β-catenin pathway and includes no discussion of the other two, more recently discovered Wnt signaling pathways or their use for treatment and/or diagnosis of glaucoma.

The second Wnt signaling pathway is the Wnt/planar cell polarity (PCP) pathway. The Wnt/PCP pathway regulates the polarity of cells through effects on the cytoskeleton. Clark et al. have reported that glaucomatous TM cells have an altered actin cytoskeletal organization (1995). It is believed that Wnt/PCP signaling operates during gastrulation and neurulation to control the movement of polarized cells. Wnt/PCP signaling plays a vital role in the appropriate orientation of trichomes, or hairs, in the adult wing of Drosophila. It is also essential for appropriate chirality of ommatidia in the Drosophila eye. It may also regulate asymmetric cell divisions of certain neuroblasts. Members of the Frizzled (Fzd) family and the cytoplasmic scaffold protein Disheveled (Dsh) function in the Wnt/PCP pathways of both the vertebrate and the invertebrate. Activity of Wnt11 is required for regulation of gastrulation movements in vertebrates. Wnt11 is thought to signal through Fzd7 to regulate protrusive activity during convergent extension. In addition to DFzd1 and Dsh, genetic studies on flies have identified a number of potential components of the Wnt/PCP pathway, including the small GTPase DrhoA, Drosophila rho-associated kinase (Drok), Fun N-terminal kinase (JNK), myosin II, myosin VIIA, and the products of the genes flamingo/starry night, fuzzy, inturned, and strabismus/van gogh.

The third Wnt signaling pathway is the Wnt/Ca²⁺ pathway. This pathway is characterized by an increase in intracellular Ca²⁺ and activation of PKC. Like the other Wnt pathways, this pathway is activated by a group of Wnt ligands and Fzd receptors distinct from those that activate other pathways, including Wnt5a, Wnt11 and Fzd2. The Wnt/Ca²⁺ pathway involves activation of a heterotrimeric G protein, elevated intracellular Ca²⁺, and activation of Ca/calmodulin kinase II and protein kinase C (PKC). It has also been shown that activation of the Wnt/Ca²⁺ pathway can antagonize the Wnt/β-catenin pathway in Xenopus, although it is unclear at what level this interaction occurs.

Diagnosing Glaucoma

Based on the inventors' finding that certain subjects with glaucoma have increased levels of Wnt/PCP pathway components or Wnt/Ca²⁺ pathway components, the present invention provides a variety of methods for diagnosing glaucoma. Certain methods of the invention can detect mutations in nucleic acid sequences that result in inappropriately high levels of Wnt/PCP pathway components or Wnt/Ca²⁺ pathway components. These diagnostics can be developed based on the known nucleic acid sequence of human Wnt/PCP pathway components or Wnt/Ca²⁺ pathway components, or the encoded amino acid sequence (see Miller 2001). Other methods can be developed based on the genomic sequence of human Wnt/PCP pathway components or Wnt/Ca²⁺ pathway components or of the sequence of genes that regulate expression of Wnt/PCP pathway components or Wnt/Ca²⁺ pathway components. Still other methods can be developed based upon a change in the level of Wnt/PCP pathway component gene expression or Wnt/Ca²⁺ pathway component gene expression at the mRNA level.

In alternative embodiments, the methods of the invention can detect the activity or level of Wnt/PCP signaling or Wnt/Ca²⁺ signaling proteins or genes encoding Wnt/PCP signaling proteins or Wnt/Ca²⁺ signaling proteins. For example, methods can be developed that detect inappropriately low Wnt/PCP signaling or Wnt/Ca²⁺ signaling activity, including for example, mutations that result in inappropriate functioning of Wnt/PCP signaling or Wnt/Ca²⁺ signaling components, including Wnt, Frizzled (Fzd), sFRP-1, Dsh, rhoA, Drok, JNK, and strabismus for PCP signaling or Ca/calmodulin kinase II (CamKII), heteromeric G protein, phospholipase C (PLC) or PKC for Ca²⁺ signaling. Methods of the invention may also be used to detect mutations that result in inappropriate functioning of Dickkopf (DKK) or LDL Receptor-Related Proteins (LRPs). In addition, non-nucleic acid based techniques may be used to detect alteration in the amount or specific activity of any of these Wnt/PCP signaling proteins or Wnt/Ca²⁺ signaling proteins.

A variety of means are currently available to the skilled artisan for detecting aberrant levels or activities of genes and gene products. These methods are well known by and have become routine for the skilled artisan. For example, many methods are available for detecting specific alleles at human polymorphic loci. The preferred method for detecting a specific polymorphic allele will depend, in part, upon the molecular nature of the polymorphism. The various allelic forms of the polymorphic locus may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. A variety of methods are available for detecting the presence of a particular single nucleotide polymorphic allele in an individual. Advancements in the field have provided accurate, easy, and inexpensive large-scale SNP genotyping. For example, see U.S. Pat. No. 4,656,127; French Patent 2,650,840; PCT App. No. WO91/02087; PCT App. No. WO92/15712; Komher et al. 1989; Sokolov 1990; Syvanen et al. 1990; Kuppuswamy et al. 1991; Prezant et al. 1992; Ugozzoli et al. 1992; Nyren et al. 1993; Roest et al. 1993; and van der Luijt et al. 1994).

Any cell type or tissue may be utilitzed to obtain nucleic acid samples for use in the diagnostics described herein. In a preferred embodiment, the DNA sample is obtained from a bodily fluid, e.g., blood, obtained by known techniques (e.g. venipuncture), saliva or tears. Most preferably, the samples for use in the methods of the present invention will be obtained from ocular tissue of the patient, such as TM cells. Alternately, nucleic acid tests can be performed on dry samples (e.g. hair or skin).

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo 1992).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allel specific hybridization using probes overlapping a region of at least one allele of a Wnt signaling component that is indicative of glaucoma and having about 5, 10, 20, 25 or 30 contiguous nucleotides around the mutation or polymorphic region. In a preferred embodiment of the invention, several probes capable of hybridizing specifically to other allelic variants involved in glaucoma are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996). In one embodiment, a chip comprises all the allelic variants of at least one polymorphic region of a gene. The solid phase support is then contacted with a test nucleic acid and hybridication to the specific probes is detected. Accordingly, the identity of numerous allelic variants of one or more genes can be identified in a simple hybridization experiment.

These techniques may further include the step of amplifying the nucleic acid before analysis. Amplification techniques are known to those of skill in the art and include, but are not limited to, cloning, polymerase chain reaction (PCR), polymerase chain reaction of is specific alleles (ASA), ligase chain reaction (LCR), nested polymerase chain reaction, self sustained sequence replication (Guatelli et al. 1990), transcriptional amplification system (Kwoh et al. 1989), and Q-Beta Replicase (Lizardi, et al. 1988).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of a Wnt/PCP signaling component or Wnt/Ca²⁺ signaling component that is indicative of glaucoma under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, aberrant levels or activities of Wnt/PCP pathway components or Wnt/Ca²⁺ pathway components that are indicative of glaucoma are identified by alterations in restriction enzyme cleavage patterns. For example, sample and control DNA is isolated, amplified (optionally), digested with one or more restriction endonucleases, and fragment length sizes are determined by gel electrophoresis.

In yet another embodiment, any of a variety of sequencing reactions known in the art can be used to directly sequence the allele. Exemplary sequencing reactions include those based on techniques developed my Maxim and Gilbert (1977) or Sanger (1977). It is also contemplated that any of a variety of automated sequencing procedures may be utilized when performing the subject assays, including sequencing by mass spectrometry (see, for example WO94/16101; Cohen et al. 1996; Griffin et al. 1993). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamin or osmium tetraoxide and with piperidine) can be used to detect mismatched bases in RNA/RNA or RNA/DNA or DNA/DNA heteroduplexes (Myers et al. 1985b; Cotton et al. 1988; Saleeba et al. 1992). In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T and G/T mismatches (Hsu et al. 1994; U.S. Pat. No. 5,459,039).

In other embodiments, alterations in electrophoretic mobility will be used to identify aberrant levels or activities of Wnt/PCP signaling pathway components or Wnt/Ca²⁺ signaling pathway components that are indicative of glaucoma. For example, single strand conformation polymorphism (SSCP) may be used to detect differences in electrophoretic mobility between mutant and wild type nucleic acids (Orita et al. 1989; Cotton 1993; Hayashi 1992; Keen et al. 1991).

In yet another embodiment, the movement of alleles in polyacrylamide gels containing a gradient of denaturant is assayed using denaturing gradient gel electrophoresis (DGGE) (Myers et al. 1985a). In a further embodiment, a temperature gradient is used in place of a denaturing agent gradient to identify differences in the mobility of control and sample DNA (Rosenbaum and Reissner 1987).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. 1986; Saiki et al. 1989). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al. 1989) or at the extreme 3′ end of one primer where, under appropriate conditions, mismatch can prevent, or reduce polymerase extension (Prossner 1993). In addition it may be desirable to introduce a novel restriction site in the region of the mutation to create cleavage-based detection (Gasparini et al. 1992). It is anticipated that in certain embodiments amplification may also be performed using Taq ligase for amplification (Barany 1991). In such cases, ligation will occur only if there is a perfect match at the 3′ end of the 5′ sequence making it possible to detect the presence of a known mutation at a specific site by looking for the presence or absence of amplification.

In another embodiment, identification of an allelic variant is carried out using an oligonucleotide ligation assay (OLA), as described, E.g., in U.S. Pat. No. 4,998,617 and in Landegren et al. 1988). Nickerson et al. have described a nucleic acid detection assay that combines attributes of PCR and OLA (Nickerson et al. 1990). In this method, PCR is used to achieve the exponential amplification of target DNA, which is then detected using OLA.

Several techniques based on this OLA method have been developed and can be used to detect aberrant levels or activities of Wnt/PCP signaling pathway components or Wnt/Ca²⁺ signaling pathway components that are indicative of glaucoma. For example, U.S. Pat. No. 5,593,826 and To be et al. (1996), describe such techniques that are frequently used.

Screening Assays for Glaucoma Therapeutics

The invention further provides screening methods for identifying glaucoma therapeutics. A glaucoma therapeutic can be any type of compound, including a protein, a peptide, peptidomimetic, small molecule, and nucleic acid. A nucleic acid can be, e.g., a gene, an antisense nucleic acid, a ribozyme, or a triplex molecule. A glaucoma therapeutic of the invention can be an agonist of a Wnt/PCP signaling pathway component activity or Wnt/Ca²⁺ signaling pathway component activity or an antagonist of FRP in the Wnt/PCP signaling pathway or in the Wnt/Ca²⁺ signaling pathway. Preferred agonists include Wnt/PCP signaling pathway components or Wnt/Ca²⁺ signaling pathway components or genes and proteins whose expression is regulated by Wnt signaling in these pathways.

The invention also provides screening methods for identifying glaucoma therapeutics which are capable of binding to an FRP protein in the Wnt/PCP signaling pathway or in the Wnt/Ca²⁺ signaling pathway or therapeutics that are capable of binding to a Wnt/PCP signaling pathway component or to a Wnt/Ca²⁺ signaling pathway component, thereby agonizing the Wnt signaling component activity.

The compounds of the invention can be identified using various assays depending on the type of compound and activity of the compound that is desired. Some examples include cell-free assays and cell-based assays. It is within the skill of the art to design additional assays for identifying glaucoma therapeutics based on the Wnt signaling based activation of trabecular meshwork genes in the Wnt/PCP signaling pathway or Wnt/Ca²⁺ signaling pathway.

Cell-free assays can be used to identify compounds which are capable of interacting with an FRP (in the Wnt/PCP signaling pathway or the Wnt/Ca²⁺ signaling pathway), Wnt/PCP signaling pathway components or Wnt/Ca²⁺ signaling pathway components, or a binding partner thereof. Such a compound can, e.g., modify the structure of an FRP, Wnt/PCP signaling pathway components or Wnt/Ca²⁺ signaling pathway components, or binding partner and thereby effect its activity. Cell-free assays can also be used to identify compounds which modulate the interaction between an FRP (in the Wnt/PCP signaling pathway or Wnt/Ca²⁺ signaling pathway), Wnt/PCP signaling pathway component or Wnt/Ca²⁺ signaling pathway component and a binding partner. In a preferred embodiment, cell-free assays for identifying such compounds consist essentially in a reaction mixture containing an FRP, Wnt/PCP signaling pathway component, or Wnt/Ca²⁺ signaling pathway component and a candidate substance or a library of candidate substances in the presence or absence of a binding partner. A candidate substance can be, e.g., a derivative of a binding partner, e.g., a biologically inactive target peptide or a small molecule.

Accordingly, one exemplary screening assay of the present invention includes the steps of contacting an FRP, Wnt/PCP signaling pathway component or Wnt/Ca²⁺ signaling pathway component, or a fragment thereof or a binding partner with a candidate substance or library of candidate substances and detecting the formation of complexes. For detection purposes, the molecule can be labeled with a specific marker and the candidate substance or library of candidate substances labeled with a different marker. The interaction of a candidate substance with an FRP, Wnt/PCP signaling pathway component or Wnt/Ca²⁺ signaling pathway component, or fragment thereof or binding partner thereof, can then be detected by determining the level of the two labels after an incubation step and a washing step. The presence of two labels after the washing step is indicative of an interaction.

Another exemplary screening assay of the present invention includes the steps of (a) forming a reaction mixture including: (i) an FRP from the Wnt/PCP signaling pathway or from the Wnt/Ca²⁺ signaling pathway, or a Wnt/PCP signaling pathway component or Wnt/Ca²⁺ signaling pathway component; (ii) a binding partner thereof; and (iii) a candidate substance; and (b) detecting interaction of the FRP from the Wnt/PCP signaling pathway or from the Wnt/Ca²⁺ signaling pathway, or a Wnt/PCP signaling pathway component or Wnt/Ca²⁺ signaling pathway component and the binding partner. The FRP from the Wnt/PCP signaling pathway or from the Wnt/Ca²⁺ signaling pathway, Wnt/PCP signaling pathway component, or Wnt/Ca²⁺ signaling pathway component and the binding partner can be produced recombinantly, purified from a source, e.g., plasma, or chemically synthesized. A statistically significant change (potentiation or inhibition) in the interaction of the FRP, Wnt/PCP signaling pathway component, or Wnt/Ca²⁺ signaling pathway component and the binding partner in the presence of the candidate substance, relative to the interaction in the absence of the candidate substance, indicates a potential agonist (mimetic or potentiator) or antagonist (inhibitor) of FRP bioactivity, Wnt/PCP signaling pathway bioactivity or Wnt/Ca²⁺ signaling pathway bioactivity for the candidate substance. The compounds of this assay can be contacted simulataneously. Alternatively, an FRP from the Wnt/PCP signaling pathway or from the Wnt/Ca²⁺ signaling pathway, Wnt/PCP signaling pathway component, or Wnt/Ca²⁺ signaling pathway component can first be contacted with a candidate substance for an appropriate amount of time, following which the binding partner is added to the reaction mixture. The efficacy of the compound can be assessed by generating dose response curves from data obtained using various concentrations of the candidate substance. Moreover, a control assay can also be performed to provide a baseline for comparison. In the control assay, isolated and purified FRP, Wnt/PCP signaling pathway component, or Wnt/Ca²⁺ signaling pathway component are added to a composition containing the FRP binding partner, Wnt/PCP signaling pathway component binding partner, or Wnt/Ca²⁺ signaling pathway component binding partner, and the formation of a complex is quantitated in the absence of the candidate substance.

Complex formation between an FRP protein and an FRP binding partner, Wnt/PCP signaling pathway component and Wnt/PCP signaling pathway component binding partner, or Wnt/Ca²⁺ signaling pathway component and Wnt/Ca²⁺ signaling pathway component binding partner may be detected by a variety of techniques. Modulation of the formation of complexes can be quantitated using, for example, detectably labeled proteins such as radiolabeled, fluorescently labeled, or enzymatically labeled FRP, Wnt/PCP signaling pathway component, Wnt/Ca²⁺ signaling pathway component or binding partners thereof, by immunoassay, or by chromatographic detection.

Typically, it will be desirable to immobilize FRP, Wnt/PCP signaling pathway component, Wnt/Ca²⁺ signaling pathway component or binding partners thereof to facilitate separation of complexes from uncomplexed forms of one or both of the proteins, as well as to accommodate automation of the assay.

For processes which rely on immunodetection for quantitating one of the proteins trapped in the complex, antibodies against the protein can be used. Alternatively, the protein to be detected in the complex can be “epitoope tagged” in the form of a fusion protein which includes, in addition to the FRP, Wnt/PCP signaling pathway component, or Wnt/Ca²⁺ signaling pathway component sequence, a second polypeptide for which antibodies are readily available (e.g. from commercial sources).

Cell-free assays can also be used to identify compounds which interact with an FRP, Wnt/PCP signaling pathway component, or Wnt/Ca²⁺ signaling pathway component and modulate their activity. Accordingly, in one embodiment, an FRP, Wnt/PCP signaling pathway component, or Wnt/Ca²⁺ signaling pathway component is contacted with a candidate substance and the catalytic activty of FRP, Wnt/PCP signaling pathway component, or Wnt/Ca²⁺ signaling pathway component is monitored. In one embodiment, the ability of FRP, Wnt/PCP signaling pathway component, or Wnt/Ca²⁺ signaling pathway component to bind to a target peptide is determined according to methods known in the art.

In addition to cell-free assays, such as described above, FRP proteins as provided by the present invention, facilitate the generation of cell-based assays, e.g., for identifying small molecule agonists or antagonists. In one embodiment, a cell expressing an FRP protein on the outer surface of its cellular membrane is incubated in the presence of a candidate substance alone or a candidate substance and a molecule which is known to interact with FRP and the interaction between FRP and a candidate substance is detected, e.g., by using a microphysiometer (McConnell et al. 1992). An interaction between the FRP protein and the candidate substance is detected by the microphysiometer as a change in the acidification of the medium. In preferred embodiments, the cell based assays of the invention utilize human cells obtained from the trabecular meshwork ocular tissue of normal or glaucoma-affected patients.

The propagation of human trabecular meshwork cells in culture allows the study of the structural and functional properties of this distinct cell type under reproducible experimental conditions. Human trabecular meshwork cells can be effectively grown from dissected explants of trabecular meshwork tissue, and the cultured cells can maintain the distinctive ultrastructural features of uncultured trabecular meshwork cells through numerous passages in vitro. The trabecular meshwork cell possesses a wide range of biochemical and structural properties that may be important for the maintenance of the aqueous outflow pathway. These properties include the growth of trabecular meshwork cells as an endothelial is monolayer with a nonthrombogenic cell surface, the production of plasminogen activator, avid phagocytosis, and the ability to synthesiz glycosaminoglycans, collagen, fibronectin, and other connective tissue elements. The presence of hyaluronidase and other lysosomal enzymes emphasizes that human trabecular meshwork cells are capable of metabolizing hyaluronic acid and other extracellular materials. Potential mechanisms of trabecular meshwork cell damage in vitro may be examined by evaluating, for example, the effects of extended passage, peroxide exposure, and laser treatment on cellular morphology.

Cell based assays based upon trabecular meshwork cells or other cell types can also be used to identify compounds which modulate expression of an FRP gene, modulate translation of an FRP mRNA, or which modulate the stability of an FRP mRNA or protein. Accordingly, in one embodiment, a cell which is capable of producing FRP, e.g., a trabecular meshwork cell, is incubated with a candidate substance and the amount of FRP produced in the cell medium is measured and compared to that produced from a cell which has not been contacted with the candidate substance. The specificity of the compound vis a vis FRP can be confirmed by various control analysis, e.g., measuring the expression of one or more control genes.

Compounds which can be tested include small molecules, proteins, and nucleic acids. In particular, this assay can be used to determine the efficacy of FRP, Wnt/PCP signaling pathway component, or Wnt/Ca²⁺ signaling pathway component antisense molecules or ribozymes.

In another embodiment, the effect of a candidate substance on transcription of an FRP gene, Wnt/PCP signaling pathway component gene, or Wnt/Ca²⁺ signaling pathway component gene is determined by transfection experiments using a reporter gene operatively linked to at least a portion of the promoter of an FRP gene, Wnt/PCP signaling pathway component gene, or Wnt/Ca²⁺ signaling pathway component gene. A promoter region of a gene can be isolated, e.g., from a genomic library according to methods known in the art. The reporter gene can be any gene encoding a protein which is readily quantifiable, e.g., the luciferase or CAT gene, well known in the art.

In a preferred embodiment, the reporter gene is a natural or synthetic gene which is transcriptionally activated in response to a Wnt/PCP pathway signal or Wnt/Ca²⁺ pathway signal.

This invention further pertains to novel agents identified by the above-described screening assays and uses thereof for treatments as described herein.

Methods of Treating Disease

A “glaucoma therapeutic,” whether an antagonist or agonist can be, as appropriate, any of the preparation described above, including isolated polypeptides, gene therapy constructs, antisense molecules, peptidomimetics, small molecules, non-nucleic acid, non-peptidic or agents identified in the drug assays provided herein.

The present invention provides for both prophylactic and therapeutic methods of treating a subject having or likely to develop a disorder associated with aberrant FRP expression or activity, Wnt/PCP signaling pathway component expression or activity, or Wnt/Ca²⁺ signaling pathway component expression or activity, e.g., glaucoma.

In one aspect, the invention provides a method for preventing in a patient (mammal), a disease or condition associated with an aberrant FRP expression or activity, Wnt/PCP signaling pathway component expression or activity, or Wnt/Ca²⁺ signaling pathway is component expression or activity by administering to the patient an agent which modulates FRP expression, Wnt/PCP signaling pathway component expression, or Wnt/Ca²⁺ signaling pathway component expression or at least one FRP activity, Wnt/PCP signaling pathway component activity, or Wnt/Ca²⁺ signaling pathway component activity. Subjects at risk for such a disease can be identified by a diagnostic or prognostic assay, e.g., as described herein. Administration of a prophylactic agent can occur prior to the manifestation of symptoms characteristic of the FRP, Wnt/PCP signaling pathway component, or Wnt/Ca²⁺ signaling pathway component aberrancy, such that a disease or disorder is prevented or, alternatively, delayed in its progression. Depending on the type of FRP, Wnt/PCP signaling pathway component, or Wnt/Ca²⁺ signaling pathway component aberrancy, for example, a FRP, Wnt/PCP signaling pathway component, or Wnt/Ca²⁺ signaling pathway component agonist or antagonist agent can be used for treating the subject prophylactically. The prophylactic methods are similar to therapeutic methods of the present invention and are further discussed below.

In general, the invention provides methods for treating a disease or condition which is caused by or contributed to by an aberrant FRP, Wnt/PCP signaling pathway component, or Wnt/Ca²⁺ signaling pathway component activity by administering to the patient or mammal an effective amount of a compound which is capable of modulating an FRP, Wnt/PCP signaling pathway component, or Wnt/Ca²⁺ signaling pathway component activity. Among the approaches which may be used to ameliorate disease symptoms involving an aberrant FRP, Wnt/PCP signaling pathway component, or Wnt/Ca²⁺ signaling pathway component activity are, for example, antisense, ribozyme, and triple helix molecules or small organic agents as described above. Examples of suitable compounds include the antagonists, agonists or homologues described in detail herein.

The agents of this invention, can be incorporated into various types of ophthalmic formulations for delivery to the eye (e.g., topically, intracamerally, or via an implant). The agents are preferably incorporated into topical ophthalmic formulations for delivery to the eye. The agents may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving an agent in a physiologically acceptable isotonic aqueous buffer. Further, the ophthalmic solution may include an ophthalmologically acceptable surfactant to assist in dissolving the agent. Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the agent in a hydrophilic base prepared from the combination of, for example, carbopol-974, or the like, according to the published formulations for analogous ophthalmic preparations; preservatives and tonicity agents can be incorporated.

The agents are preferably formulated as topical ophthalmic suspensions or solutions, with a pH of about 4 to 8. The establishment of a specific dosage regimen for each individual is left to the discretion of the clinicians. The agents will normally be contained in these formulations in an amount 0.01% to 5% by weight, but preferably in an amount of 0.05% to 2% and most preferably in an amount 0.1 to 1.0% by weight. The dosage form may be a solution, suspension microemulsion. Thus, for topical presentation 1 to 2 drops of these formulations would be delivered to the surface of the eye 1 to 4 times per day according to the discretion of a skilled clinician.

The agents can also be used in combination with other agents for treating glaucoma, such as, but not limited to, β-blockers, prostaglandin analogs, carbonic anhydrase inhibitors, α₂ agonists, miotics, and neuroprotectants.

The agent may be delivered directly to the eye (for example: topical ocular drops or ointments; slow release devices in the cul-de-sac or implanted adjacent to the sclera or within the eye; periocular, conjunctival, sub-Tenons, intracameral or intravitreal injections) or parenterally (for example: orally; intravenous, subcutaneous or intramuscular injections; dermal delivery; etc.) using techniques well known by those skilled in the art.

It is further contemplated that the agents of the invention can be formulated in intraocular insert devices.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the is invention.

### EXAMPLE 1

Wnt Ca²⁺ Pathway: Calcium Mobilization Assay

To test if a compound affect calcium mobilization, human TM cells grown on #0 coverslips are loaded with a calcium fluorescent dye, fura-2 acetoxymethyl ester for 60 minutes at room temperature in Hepes buffer containing: NaCl 125 mM, KCl 5 mM, CaCl₂ 1.8 mM, MgCl₂ 2 mM, NaH₂PO₄ 0.5 mM, NaHCO₃ 5 mM, glucose 10 mM, bovine serum albumin 0.1%, and Hepes 10 mM, pH 7.2. After the incubation, the coverslip is rinsed with the same buffer without the dye and mounted in a chamber on the stage of a microscope. Intracellular calcium can be monitored by various ratiofluorometric methods and systems, such as the DeltaScan 4000 ratio fluorescence System (Photon Technology International). Treating the cells with compounds that acutely increase the Wnt activity should increase intracellular calcium concentration after a short period of treatment. Compounds that decrease Wnt activity are expected to decrease intracellular calcium acutely. For compounds that affect the expression of FRP-1 or Wnt proteins, the cells should be treated with these compounds for 24 hours or longer and their intracellular calcium compared to the untreated cells.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Patents
U.S. Pat. No. 4,656,127
U.S. Pat. No. 4,998,617
U.S. Pat. No. 5,459,039
U.S. Pat. No. 5,593,826

Foreign Patents and Published Patent Applications
French Patent 2,650,840
WO91/02087
WO92/15712
WO94/16101

Books
Nuovo, G. J., PCR in situ HYBRIDIZATION: PROTOCOLS AND APPLICATIONS, Raven Press, NY (1992).

Other Publications
Barany, Proc. Natl. Acad. Sci. USA 88: 189 (1991).
Clark et al., need cite
Cohen et al. Adv. Chromatog. 36: 127-162 (1996).
Cotton et al., Proc. Natl. Acad. Sci. USA 85: 4397 (1988).
Cotton, Mutat. Res. 285: 125-144 (1993).
Cronin et al., Human Mutation 7: 244 (1996).
Deardorff and Klein, Mech. Dev. 87: 229 (1999).
Gasparini et al., Mol. Cell Probes 6: 1 (1992).
Gibbs et al., Nucleic Acids Res. 17: 2437-2448 (1989).
Griffin et al., Appl. Biochem Biotechnol. 38: 147-159 (1993).
Guatelli, J. C., et al., Proc. Natl. Acad. Sci. USA 87: 1874-1878 (1990).
Hahn et al., Cell 85: 841 (1996).
Hayashi, Genet. Anal. Tech. Appl. 9: 73-79 (1992).
Hsu et al., Carcinogenesis 15: 1657-1662 (1994).
Ingham, Curr. Biol. 4: 1 (1994).
Jasoni et al., Dev. Dyn. 215: 215 (1999).
Johnson et al., Science 272: 1668 (1996).
Keen et al., Trends Genet. 7: 5 (1991).
Komher, J. S. et al., Nucl. Acids Res. 17: 7779-7784 (1989).
Ku and Melton, Development 119: 1161 (1993).
Kuppuswamy, M. N. et al. Proc. Natl. Acad. Sci. (U.S.A.) 88: 1143-1147 (1991).
Kwoh, D. Y., et al., Proc. Natl. Acad. Sci. USA 86: 1173-1177 (1989).
Landegren, U. et al., Science 241: 1077: 1080 (1988).
Lizardi, P. M. et al., Bio/Technology 6: 1197 (1988).
Maxim and Gilbert, Proc. Natl. Acad. Sci. USA 74: 560 (1977).
McConnell et al., Science 257: 1906 (1992).
McMahon and Bradley, Cell 62: 1073 (1990).
Miller, Genome Biology 3(1):reviews 3001.1-3001.15 (2001) (also at http://genomebiology.com/2001/3/1/reviews/3001.1).
Myers et al., Nature 313: 495 (1985a).
Myers et al., Science 230: 1242 (1985b).
Nickerson, D. A. et al, Proc. Natl. Acad. Sci USA 87: 8923-27 (1990).
Niswander et al., Nature 371: 609 (1994).
Nyren, P. et al. Anal. Biochem. 208: 171-175 (1993).
Orita et al., Proc. Natl. Acad. Sci. USA 86: 2766 (1989).
Prezant, T. R. et al., Hum. Mutat. 1: 159-164 (1992).
Prossner, Tibtech 11: 238 (1993).
Roest et al., Hum. Mol. Genet. 2: 1717-1721 (1993).
Rosenbaum and Reissner, Biophys. Chem. 265: 12753 (1987).
Saiki et al., Nature 324: 163 (1986).
Saiki et al., Proc. Natl. Acad. Sci. USA 86: 6230 (1989).
Saleeba et al., Methods Enzymol. 217: 286-295 (1992).
Sanger et al. Proc. Natl. Acad. Sci. USA 74: 5463 (1977).
Sokolov, B. P., Nucl. Acids Res. 18: 3671 (1990).
Stern et al., Development 121: 3675 (1995).
Syvanen, A.-C., et al., Genomics 8: 684-692 (1990).
Tobe et al., Nucleic Acids Res. 24: 3728 (1996).
Ugozzoli, L. et al., GATA 9: 107-112 (1992).
Van der Luijt et al., Genomics 20: 1-4 1994).
Wilder and Perrimon, Development 121: 477 (1995).

1. A method for diagnosing glaucoma in a patient, said method comprising the steps of:
(a) obtaining a sample from said patient;
(b) detecting the level or bioactivity of Wnt/Ca²⁺ pathway component, a frizzled related protein gene product of the Wnt/Ca²⁺ pathway, or an FRP of the Wnt/Ca²⁺ pathway; and
(c) comparing the level or bioactivity of Wnt/Ca²⁺ pathway component, frizzled related protein gene product of the Wnt/Ca²⁺ pathway, or FRP of the Wnt/Ca²⁺ pathway with the level in a normal sample;
wherein an aberrant level or bioactivity of Wnt/Ca²⁺ pathway component, a frizzled related protein gene product of the Wnt/Ca²⁺ pathway, or an FRP of the Wnt/Ca²⁺ pathway is indicative of a glaucomatous state.

2. The method of claim 1, wherein the patient sample comprises cells of the trabecular meshwork tissue or patient tears.

3. A method for diagnosing glaucoma in a patient, said method comprising the steps of:
(a) obtaining a sample from said patient;
(b) isolating a Wnt/Ca²⁺ pathway component, a frizzled related protein gene product of the Wnt/Ca²⁺ pathway, or an FRP of the Wnt/Ca²⁺ pathway from said sample; and
(c) comparing the sequence of Wnt/Ca²⁺ pathway component, frizzled related protein gene product of the Wnt/Ca²⁺ pathway, or FRP of the Wnt/Ca²⁺ pathway obtained from the sample with the sequence of a wildtype Wnt/Ca²⁺ pathway component, frizzled related protein gene product of the Wnt/Ca²⁺ pathway, or FRP of the Wnt/Ca²⁺ pathway;
wherein the presence of a genetic lesion in the sequence of Wnt/Ca²⁺ pathway component, frizzled related protein gene product of the Wnt/Ca²⁺ pathway, or FRP of the Wnt/Ca²⁺ pathway obtained from said sample as compared to the wildtype sequence indicates a glaucomatous state.

4. A method of identifying an agent potentially useful for treating glaucoma, said method comprising the steps of:
(a) contacting a cell expressing Wnt/Ca²⁺ pathway component with a candidate substance;
(b) detecting a level or bioactivity of said Wnt/Ca²⁺ pathway component in the presence of the candidate substance; and
(c) comparing the level or bioactivity of said Wnt/Ca²⁺ pathway component in the presence of the candidate substance with that in the absence of the candidate substance;
wherein an increase in the level or bioactivity of the Wnt/Ca²⁺ pathway component in the presence of the candidate substance as compared to the level or bioactivity detected in the absence of the candidate substance identifies said candidate substance as an agent potentially useful for treating glaucoma.

5. A method of identifying an agent potentially useful for treating glaucoma, said method comprising the steps of:
(a) admixing a composition comprising a Wnt/Ca²⁺ pathway component polypeptide with a candidate substance;
(b) adding a composition comprising a Wnt/Ca²⁺ pathway component binding partner to the solution obtained in step (a) under conditions conducive to allow binding of the Wnt/Ca²⁺ pathway component polypeptide to the Wnt/Ca²⁺ pathway component binding partner;
(c) detecting the interaction of the Wnt/Ca²⁺ pathway component polypeptide with the binding partner; and
(d) comparing the interaction of the Wnt/Ca²⁺ pathway component polypeptide and the binding partner in the presence of the candidate substance with that in the absence of said candidate substance;
wherein an increase or decrease in the interaction of the Wnt/Ca²⁺ pathway component polypeptide with the binding partner in the presence of the candidate substance as compared to that in the absence of the candidate substance identifies the candidate as an agent potentially useful for treating glaucoma.

6. The method of claim 5, wherein the Wnt/Ca²⁺ pathway component is selected from the group consisting of LRP, Fzd, heteromeric G protein, PLC, PKC, CamKII and FRP.

7. A method for treating glaucoma in a patient, said method comprising administering to said patient a composition comprising a therapeutically effective amount of a compound that modulates the level or bioactivity of a Wnt/Ca²⁺ pathway component, a frizzled related protein gene product of the Wnt/Ca²⁺ pathway, or an FRP of the Wnt/Ca²⁺ pathway.

8. The method of claim 7, wherein the compound is selected from the group consisting of a protein, a peptide, a peptidomimetic, a small molecule or a nucleic acid.

9. The method of claim 8, wherein the nucleic acid is selected from the group consisting of a gene, antisense, ribozyme and triplex nucleic acid.

10. A composition for treating glaucoma comprising a therapeutically effective amount of a compound that modulates the level or bioactivity of a Wnt/Ca²⁺ pathway component, a frizzled related protein gene product of the Wnt/Ca²⁺ pathway, or an FRP of the Wnt/Ca²⁺ pathway.
